(19) United States
(12) Patent Application Publication
(10) Pub. No.: US 2023/0225620 A1
(43) Pub. Date: Jul. 20, 2023

(54) DETECTION OF ELEVATED BODY TEMPERATURE USING CIRCADIAN RHYTHMS SYSTEMS AND METHODS

(71) Applicant: FLIR Systems AB, Täby (SE)

(72) Inventors: Jonas Sandsten, Täby (SE); Nicklas Bahram Ramberg, Täby (SE)

(21) Appl. No.: 18/184,608

(22) Filed: Mar. 15, 2023

Related U.S. Application Data
(63) Continuation of application No. PCT/US2021/051484, filed on Sep. 22, 2021.
(60) Provisional application No. 63/082,385, filed on Sep. 23, 2020.

(51) Int. Cl. A61B 5/01; A61B 5/00; G16H 10/60; G16H 50/30

(52) U.S. Cl. CPC A61B 5/015; A61B 5/4806; A61B 5/746; A61B 5/7267; G16H 10/60; G16H 50/30

(57) ABSTRACT

Various techniques are disclosed to provide for improved detection of elevated human body temperatures. In one example, a method includes receiving a thermal image. The method also includes processing the thermal image to detect a person's face and a characteristic associated with the person. The method also includes selecting a circadian rhythm model associated with the detected characteristic.

The method also includes determining an expected body temperature using the circadian rhythm model. The method also includes extracting a temperature associated with the person's face from the thermal image. The method also includes comparing the extracted temperature with the expected body temperature to detect an elevated body temperature condition. Additional methods and systems are also provided.

### CROSS-REFERENCE TO RELATED APPLICATIONS

This application is a continuation of International Patent Application No. PCT/US2021/051484 filed Sep. 22, 2021 and entitled “DETECTION OF ELEVATED BODY TEMPERATURE USING CIRCADIAN RHYTHMS SYSTEMS AND METHODS,” which claims the benefit of and priority to U.S. Provisional Patent Application No. 63/082,385 filed Sep. 23, 2020 and entitled “DETECTION OF ELEVATED BODY TEMPERATURE USING CIRCADIAN RHYTHMS SYSTEMS AND METHODS,” all of which are incorporated herein by reference in their entirety.

### TECHNICAL FIELD

The present invention relates generally to thermal imaging and, more particularly, to detecting elevated body temperature with thermal imaging.

### BACKGROUND

Thermal imaging systems are frequently used to detect the temperatures of various objects or persons in a scene. For example, in the case of human beings, such systems may be used to detect body temperature. Such systems can be particularly useful in the detection of elevated body temperatures associated with possible health conditions (e.g., infections or disease). For example, if the detected body temperature of a person is found to exceed a predetermined threshold, then the person may be identified as having an elevated body temperature and subject to further review for a possible health condition.

Conventional detection systems typically rely on predetermined threshold temperatures. However, such approaches can fail to account for natural variations in body temperature among different persons that are not associated with health conditions. For example, persons may exhibit different body temperatures due to natural variations associated with differences in circadian rhythms, thermo-regulation, and/or physical exertion (e.g., exercise).

Some detection systems may rely on a moving average of temperature values measured from random groups of people. However, the moving average may not be useful in circumstances where the group is diverse with persons of different ages or other characteristics (e.g., children, post-menopause, seniors, or others). In these cases, the moving average may be applicable to only a subset of the group and may not yield helpful results when applied to individual persons that vary significantly from the average for non-health related reasons.

### SUMMARY

Various techniques are disclosed to provide for improved detection of elevated human body temperatures. In some embodiments, such techniques may classify persons into groups associated with certain circadian rhythms so as to perform an accurate comparison of a person's body temperature to elevated body temperature threshold values associated with the group. In some embodiments, temperature values (e.g., temperature measurements) associated with multiple facial regions of a person may be extracted from thermal images and compared to provide an accurate measurement of the person's body temperature. Additional fever detection techniques are also provided.

In one embodiment, a method includes receiving a thermal image; processing the thermal image to detect a person's face and a characteristic associated with the person; selecting a circadian rhythm model associated with the detected characteristic; determining an expected body temperature using the circadian rhythm model; extracting a temperature associated with the person's face from the thermal image; and comparing the extracted temperature with the expected body temperature to detect an elevated body temperature condition.

In another embodiment, a system includes a thermal imager; and a logic device configured to: operate the thermal imager to capture a thermal image, process the thermal image to detect a person's face and a characteristic associated with the person, select a circadian rhythm model associated with the detected characteristic, determine an expected body temperature using the circadian rhythm model, extract a temperature associated with the person's face from the thermal image, and compare the extracted temperature with the expected body temperature to detect an elevated body temperature condition.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 illustrates a block diagram of an imaging system in accordance with an embodiment of the disclosure.

FIG. 2 illustrates a block diagram of a thermal imager in accordance with an embodiment of the disclosure.

FIG. 3 illustrates a block diagram of an artificial neural network in accordance with an embodiment of the disclosure.

FIG. 4 illustrates a thermal image undergoing processing in accordance with an embodiment of the disclosure.

FIG. 5 illustrates a process of detecting elevated body temperature using circadian rhythms in accordance with an embodiment of the disclosure.

Embodiments of the present invention and their advantages are best understood by referring to the detailed description that follows. It should be appreciated that like reference numerals are used to identify like elements illustrated in one or more of the figures.

### DETAILED DESCRIPTION

In accordance with embodiments discussed herein, various methods and systems are provided in which thermal images are processed to determine elevated body temperatures of human beings (e.g., persons) using a heuristic approach that accounts for temperature variances associated with the particular circadian rhythms of persons of different ages, genders, and/or other characteristics.

Circadian rhythms are processes within living organisms that repeat in cycles approximately every 24 hours. For example, human body temperature can vary over the course of each circadian rhythm cycle and can be affected by age, gender, and other characteristics associated with a person. In some cases, the average cosinor amplitude of a person's body temperature can vary as much as 2 degrees C. over the course of each circadian rhythm cycle. Indeed, some individuals may experience even larger variations as high as 3 or 4 degrees C.

By classifying persons into groups with shared associated characteristics that affect their circadian rhythms, the body temperature of a person in the group may be compared with temperatures (e.g., moving average temperatures or other measurements) of other persons in the group having the shared associated characteristics (e.g., and consequently the same or similar expected circadian rhythm temperature variations). As a result, elevated body temperature can be detected more accurately over conventional approaches that utilize temperatures (e.g., moving average temperatures or other measurements) associated with random collections of persons with dissimilar characteristics.

In some embodiments, an artificial neural network (e.g., a convolution neural network in some cases) may be used to process images of a person captured by a thermal imaging system (e.g., a thermal imaging camera). For example, the neural network may be used to detect the person's face and various characteristics such as age, gender, and/or others. In some embodiments, the neural network may process one or more thermal images captured by the thermal imaging system of persons passing through a scene imaged by the thermal imaging system. In some embodiments, the one or more thermal images may be converted to corresponding visible light representations of the thermal images before being processed by the neural network.

As discussed, circadian rhythms can be affected by a person's age, gender, and other characteristics. In this regard, groups of persons having shared characteristics (e.g. having the same gender, an age within a particular range of ages, and/or other shared characteristics) may share similar variations in body temperature over the course of their circadian rhythm cycles. Accordingly, by comparing a person's body temperature with the body temperatures of a group of persons having similar circadian rhythm cycles, and also determining the time (e.g., to determine the expected phase of the circadian rhythm cycle), it can be accurately determined whether the person's body temperature is elevated relative to the group. Such an approach can provide an accurate detection of elevated body temperature that correctly accounts for individual characteristics of the person.

In some embodiments, a fever may be detected in a person by using temperature values taken from two or more sites on the person's face, such as one or both bilateral canthi regions and/or an oral region (e.g., mouth). Such an approach can be useful for detecting potential fever in a person, particularly if that person's body temperature falls outside of expected circadian rhythm body temperature variations associated with other persons having the same or similar shared characteristics. In some cases, a fever may be detected with increased probability over conventional approaches if one or more of the following conditions are met: a difference in temperature between the inner canthus regions is lower than a fixed value (e.g., less than 0.2 degrees C.); a difference in temperature between one of the inner canthus regions and the oral region is less than another fixed value (e.g., less than 0.5 degrees C.); and/or an absolute value of the average values of both the inner canthus regions and the oral region is above 37 degrees C.

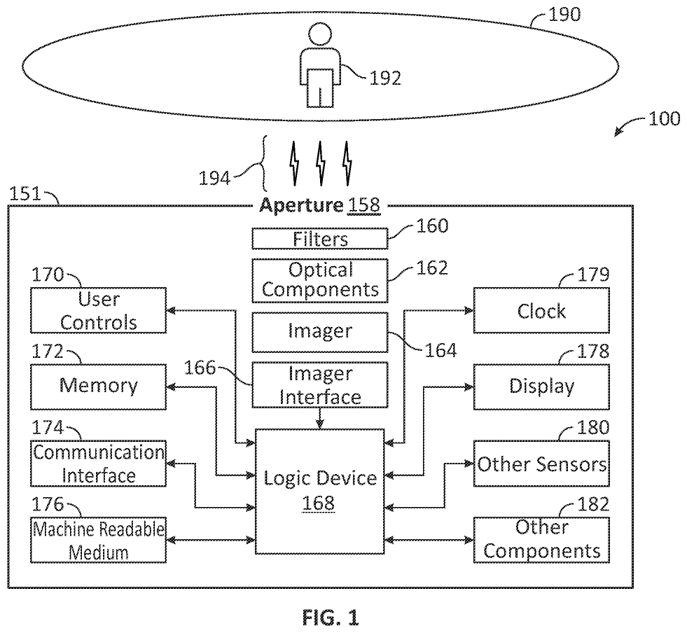

Turning now to the drawings, FIG. 1 illustrates a block diagram of an imaging system 100 in accordance with an embodiment of the disclosure. As shown, imaging system 100 includes a housing 151 (e.g., a camera body) having an aperture 158, one or more filters 160, one or more optical components 162, a thermal imager 164, an imager interface 166, a logic device 168, user controls 170, a memory 172, a communication interface 174, a machine readable medium 176, a display 178, a clock 179, other sensors 180, and other components 182.

In various embodiments, imaging system 100 may be implemented, for example, as a camera system such as a portable (e.g., handheld) thermal camera system, a small form factor camera system implemented as part of another device, a fixed camera system, and/or other appropriate implementations. Imaging system 100 may be positioned to receive infrared radiation 194 from a scene 190 (e.g., a field of view of imaging system 100). In various embodiments, scene 190 may include various features of interest such as one or more persons 192 (e.g., human beings).

Clock 179 may be implemented as any appropriate type of device used to measure time of day. In some embodiments, such time measurements may be used to determine the current phase of a circadian rhythm associated with person 192 to detect a possible elevated body temperature.

Infrared radiation 194 is received through aperture 158 and passes through one or more filters 160 which may be provided to selectively filter particular thermal wavelengths of interest for images to be captured by thermal imager 164. Optical components 162 (e.g., an optical assembly including one or more lenses, additional filters, transmissive windows, and/or other optical components) pass the filtered infrared radiation 194 for capture by thermal imager 164.

Thus, it will be appreciated that filters 160 and/or optical components 162 may operate together to selectively filter out portions of infrared radiation 194 such that only desired wavelengths and/or desired thermal radiation intensities are ultimately received by thermal imager 164. In various embodiments, any desired combination of such components may be provided (e.g., various components may be included and/or omitted as appropriate for various implementations).

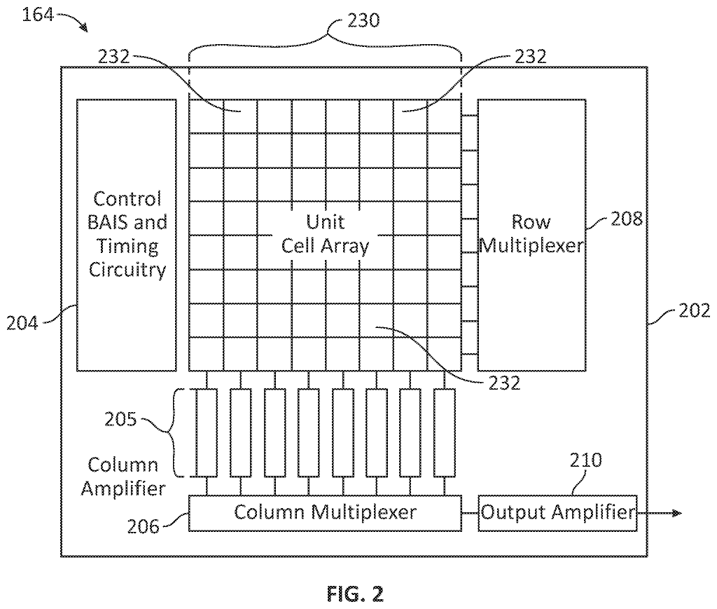

Thermal imager 164 may capture thermal images of scene 190 in response to infrared radiation 194. Thermal imager 164 may include an array of sensors for capturing thermal images (e.g., thermal image frames) of scene 190. In some embodiments, thermal imager 164 may also include one or more analog-to-digital converters for converting analog signals captured by the sensors into digital data (e.g., pixel values) to provide the captured images. Imager interface 166 provides the captured images to logic device 168 which may be used to process the images, store the original and/or processed images in memory 172, and/or retrieve stored images from memory 172. Additional implementation details of an embodiment of thermal imager 164 are discussed herein with regard to FIG. 2.

Logic device 168 may include, for example, a microprocessor, a single-core processor, a multi-core processor, a microcontroller, a programmable logic device configured to perform processing operations, a digital signal processing (DSP) device, one or more memories for storing executable instructions (e.g., software, firmware, or other instructions), and/or any other appropriate combinations of devices and/or memory to perform any of the various operations described herein. Logic device 168 is configured to interface and communicate with the various components of imaging system 100 to perform various method and processing steps described herein. In various embodiments, processing instructions may be integrated in software and/or hardware as part of logic device 168, or code (e.g., software and/or configuration data) which may be stored in memory 172 and/or a machine readable medium 176. In various embodiments, the instructions stored in memory 172 and/or machine readable medium 176 permit logic device 168 to perform the various operations discussed herein and/or control various components of system 100 for such operations.

Memory 172 may include one or more memory devices (e.g., one or more memories) to store data and information. The one or more memory devices may include various types of memory including volatile and non-volatile memory devices, such as RAM (Random Access Memory), ROM (Read-Only Memory), EEPROM (Electrically-Erasable Read-Only Memory), flash memory, fixed memory, removable memory, and/or other types of memory.

Machine readable medium 176 (e.g., a memory, a hard drive, a compact disk, a digital video disk, or a flash memory) may be a non-transitory machine readable medium storing instructions for execution by logic device 168. In various embodiments, machine readable medium 176 may be included as part of imaging system 100 and/or separate from imaging system 100, with stored instructions provided to imaging system 100 by coupling the machine readable medium 176 to imaging system 100 and/or by imaging system 100 downloading (e.g., via a wired or wireless link) the instructions from the machine readable medium (e.g., containing the non-transitory information).

Logic device 168 may be configured to process captured images and provide them to display 178 for presentation to and viewing by the user. Display 178 may include a display device such as a liquid crystal display (LCD), an organic light-emitting diode (OLED) display, and/or other types of displays as appropriate to display images and/or information to the user of system 100. Logic device 168 may be configured to display images and information on display 178. For example, logic device 168 may be configured to retrieve images and information from memory 172 and provide images and information to display 178 for presentation to the user of system 100. Display 178 may include display electronics, which may be utilized by logic device 168 to display such images and information.

User controls 170 may include any desired type of user input and/or interface device having one or more user actuated components, such as one or more buttons, slide bars, knobs, keyboards, joysticks, and/or other types of controls that are configured to generate one or more user actuated input control signals. In some embodiments, user controls 170 may be integrated with display 178 as a touchscreen to operate as both user controls 170 and display 178. Logic device 168 may be configured to sense control input signals from user controls 170 and respond to sensed control input signals received therefrom. In some embodiments, portions of display 178 and/or user controls 170 may be implemented by appropriate portions of a tablet, a laptop computer, a desktop computer, and/or other types of devices.

In various embodiments, user controls 170 may be configured to include one or more other user-activated mechanisms to provide various other control operations of imaging system 100, such as auto-focus, menu enable and selection, field of view (FoV), brightness, contrast, gain, offset, spatial, temporal, and/or various other features and/or parameters.

Imaging system 100 may include various types of other sensors 180 including, for example, microphones, navigation sensors, temperature sensors, and/or other sensors as appropriate.

Logic device 168 may be configured to receive and pass images from imager interface 166 and signals and data from clock 179, sensors 180, and/or user controls 170 to one or more external devices (e.g., remote systems) through communication interface 174 (e.g., through wired and/or wireless communications). In this regard, communication interface 174 may be implemented to provide wired communication over a cable and/or wireless communication over an antenna. For example, communication interface 174 may include one or more wired or wireless communication components, such as an Ethernet connection, a wireless local area network (WLAN) component based on the IEEE 802.11 standards, a wireless broadband component, mobile cellular component, a wireless satellite component, or various other types of wireless communication components including radio frequency (RF), microwave frequency (MWF), and/or infrared frequency (IRF) components configured for communication with a network. As such, communication interface 174 may include an antenna coupled thereto for wireless communication purposes. In other embodiments, the communication interface 174 may be configured to interface with a DSL (e.g., Digital Subscriber Line) modem, a PSTN (Public Switched Telephone Network) modem, an Ethernet device, and/or various other types of wired and/or wireless network communication devices configured for communication with a network.

In some embodiments, a network may be implemented as a single network or a combination of multiple networks. For example, in various embodiments, the network may include the Internet and/or one or more intranets, landline networks, wireless networks, and/or other appropriate types of communication networks. In another example, the network may include a wireless telecommunications network (e.g., cellular phone network) configured to communicate with other communication networks, such as the Internet. As such, in various embodiments, imaging system 100 and/or its individual associated components may be associated with a particular network link such as for example a URL (Uniform Resource Locator), an IP (Internet Protocol) address, and/or a mobile phone number.

Imaging system 100 may include various other components 182 such as speakers, additional displays, visual indicators (e.g., recording indicators), vibration actuators, a battery or other power supply (e.g., rechargeable or otherwise), and/or additional components as appropriate for particular implementations.

In some embodiments, imaging system 100 may include hardware (e.g., implemented in optical components 162, imager 164, and/or other components 192) and/or software (e.g., implemented in logic device 168 and/or machine readable medium 176) to perform image stabilization and/or averaging of spatial and temporal pixel values associated with particular facial features of person 192.

Although various features of imaging system 100 are illustrated together in FIG. 1, any of the various illustrated components and subcomponents may be implemented in a distributed manner and used remotely from each other as appropriate (e.g., through appropriate wired and/or wireless network communication).

Although imaging system 100 has been described in the context of a thermal imaging system, other embodiments are also contemplated. In some embodiments, aperture 158, filters 160, optical components 162, and/or imager 164 may be implemented to pass and capture other wavelengths such as visible light wavelengths in addition to or instead of thermal wavelengths. For example, imaging system 100 may be implemented to capture both thermal images and visible light images of scene 190 for comparison with each other to detect scaling or other phenomena. As another example, different imaging systems 100 implemented for different wavelengths may be used to capture thermal images and visible light images of scene 190.

FIG. 2 illustrates a block diagram of thermal imager 164 in accordance with an embodiment of the disclosure. In this illustrated embodiment, thermal imager 164 is a focal plane array (FPA) including a sensor array 230 of infrared sensors 232 (e.g., implemented as unit cells) and a read out integrated circuit (ROIC) 202. Although an 8 by 8 array of infrared sensors 232 is shown (e.g., corresponding to rows and columns of pixels), this is merely for purposes of example and ease of illustration. Any desired sensor array size may be used as desired.

Each infrared sensor 232 may be implemented, for example, by an infrared detector such as a microbolometer and associated circuitry to provide image data (e.g., a data value associated with a captured voltage) for a pixel of a captured thermal image. In this regard, time-multiplexed electrical signals may be provided by the infrared sensors 232 to ROIC 202.

ROIC 202 includes bias generation and timing control circuitry 204, column amplifiers 205, a column multiplexer 206, a row multiplexer 208, and an output amplifier 210. Images captured by infrared sensors 232 may be provided by output amplifier 210 to logic device 168 and/or any other appropriate components to perform various processing techniques described herein. Further descriptions of ROICs and infrared sensors (e.g., microbolometer circuits) may be found in U.S. Pat. No. 6,028,309 issued Feb. 22, 2000, which is incorporated herein by reference in its entirety.

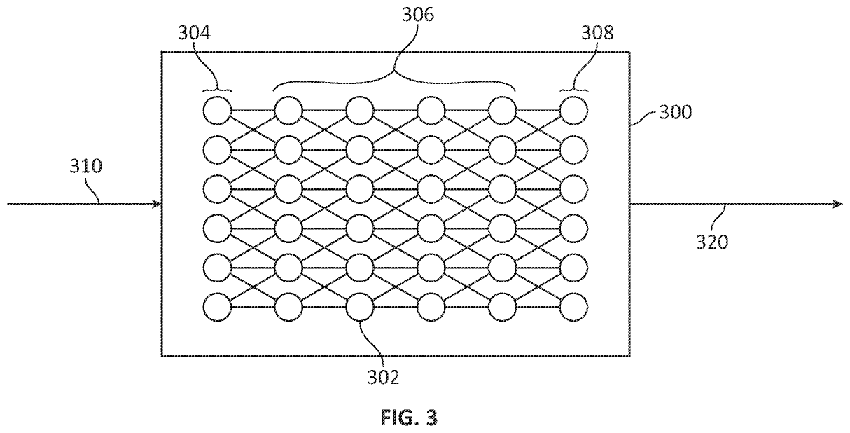

FIG. 3 illustrates a block diagram of an artificial neural network 300 in accordance with an embodiment of the disclosure. For example, in some embodiments, neural network 300 may be implemented by logic device 168. As discussed, neural network 300 (e.g., a convolution neural network in some cases) may be used to process images of person 192 to detect their face and additional characteristics such as age, gender, and/or other characteristics.

As shown, neural network 300 includes various nodes 302 (e.g., neurons) arranged in multiple layers including an input layer 304 receiving one or more inputs 310, hidden layers 306, and an output layer 308 providing one or more outputs 320. Although particular numbers of nodes 302 and layers 304, 306, and 308 are shown, any desired number of such features may be provided in various embodiments.

In some embodiments, neural network 300 may be used to perform face detection and additional characteristic detection on various thermal images captured by imaging system 100 and provided to inputs 310 of neural network 300. The results of such detection may be provided by neural network 300 at outputs 320. In some embodiments, neural network 300 may be trained by providing thermal and/or visible light images of known human faces with known characteristics (e.g., images and related information regarding the characteristics stored in machine readable medium 176 and/or received through communication interface 174) to inputs 310.

In some embodiments, neural network 300 operates as a multi-layer classification tree using a set of non-linear transformations between the various layers 304, 306, and/or 308 to extract features and information from thermal images captured by imager 164 (or visible images generated therefrom in some embodiments). For example, neural network 300 may be trained on large amounts of data (e.g., thermal or visible images of human faces) such that it learns to distinguish human characteristics. This iterative procedure is repeated until neural network 300 has trained on enough data such that it can perform predictions of its own.

In some embodiments, facial recognition may be performed by neural network 300 detecting facial features such as eye spacing, nose width, eye hole depth, jaw width, and/or other features. In some embodiments, age may be determined by neural network 300 detecting actual age, appearance age, apparent age, and/or estimated age.

As discussed, in some embodiments, neural network 300 may be implemented as a convolution neural network. For example, in such a convolution neural network, one or more of layers 304, 306, and/or 308 may be implemented as convolution layers, pooling layers, and/or fully connected layers. In this regard, a convolution layer provides a representation of various features to be detected by utilizing several convolution kernels where a single node 302 in a feature map is connected to a set of nodes 302 in a feature of a previous layer. A pooling layer obtains shift invariance by reducing the resolution of the feature map and is typically implemented between two convolution layers. A fully connected layer uses all the nodes 302 in a previous layer and connects them to every node 302 in the fully connected layer.

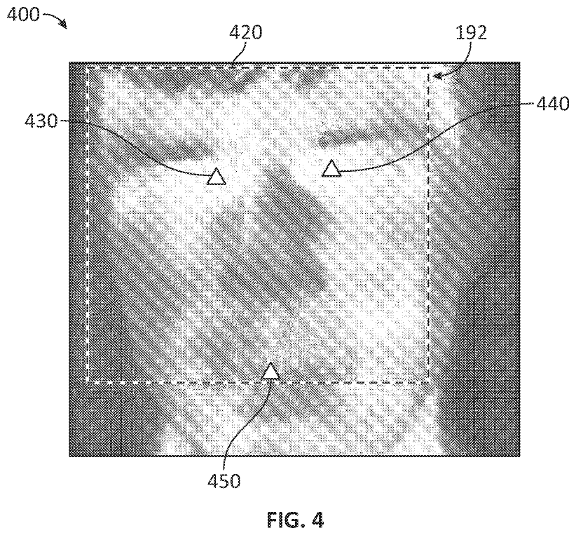

FIG. 4 illustrates a thermal image 400 undergoing processing in accordance with an embodiment of the disclosure. For example, thermal image 400 may be an image of person 192 captured by imager 164. In this regard, imager 164 may be implemented with sufficient resolution to capture individual features (e.g., corresponding to appropriate spot size ratios) on a face 420 of person 192. As shown, face 420 of person 192 has been detected (e.g., denoted by a square perimeter superimposed on thermal image 400) by neural network 300 performing appropriate processing. As discussed herein, neural network 300 may perform additional processing as appropriate to detect the age, gender, and/or other characteristics of person 192.

As also shown in FIG. 4, several temperature measurement locations (e.g., sites) 430, 440, and 450 are identified. In this regard, locations 430 and 440 correspond to the left inner canthus and the right inner canthus, respectively, of person 192. Location 450 corresponds to an oral region (e.g., mouth or throat) of person 192. Temperature values may be extracted from one or more of locations 430, 440, and/or 450 of thermal image 400 which correlate to body temperature to determine whether person 192 exhibits an elevated body temperature and/or a fever.

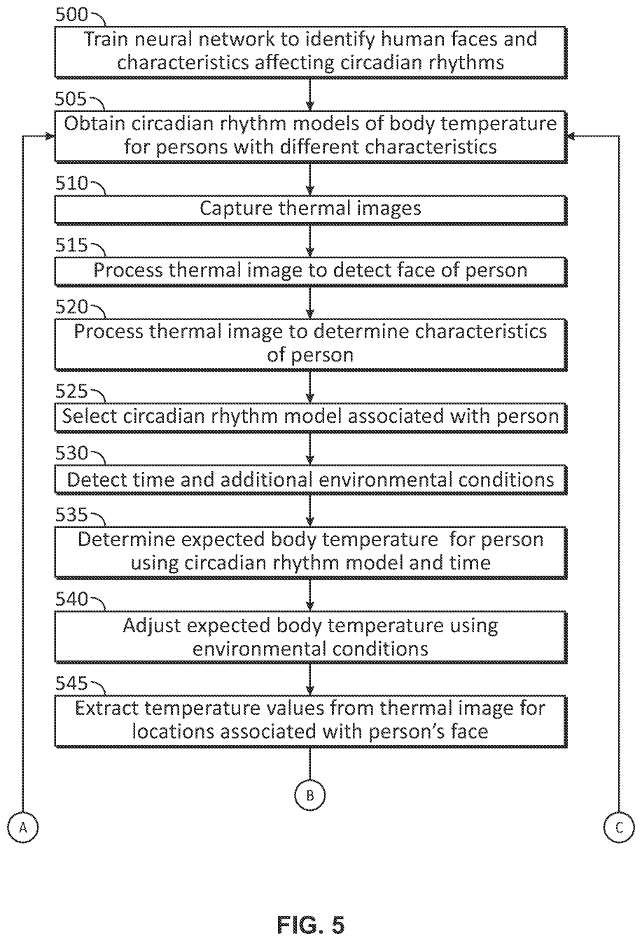

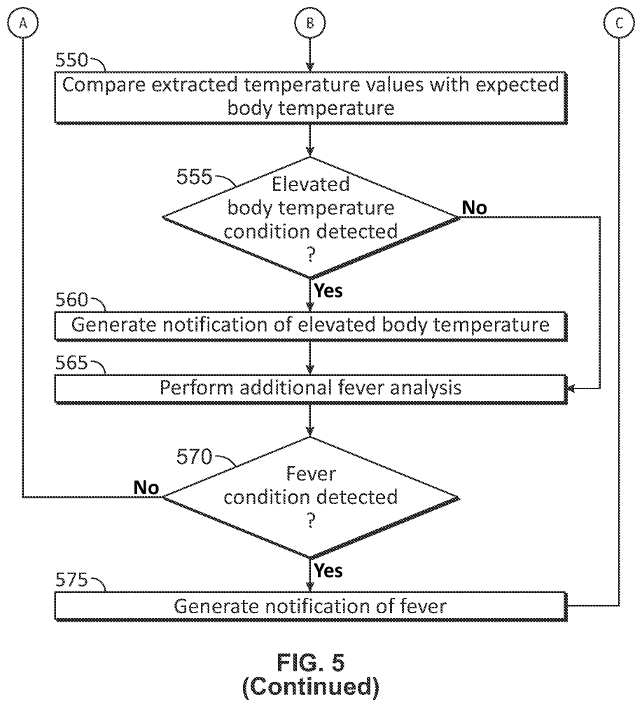

As discussed, imaging system 100 may include hardware and/or software to perform image stabilization and/or averaging of spatial and temporal pixel values associated with one or more of locations 430, 440, and/or 450 to improve the accuracy of temperature values extracted from a plurality of thermal images 400. Additional aspects of the processing of thermal image 400 are discussed with regard to the process of FIG. 5.

FIG. 5 illustrates a process of detecting elevated body temperature using circadian rhythms in accordance with an embodiment of the disclosure. In various embodiments, the actions discussed with regard to FIG. 5 may be performed by appropriate portions of imaging system 100 and/or other systems in communication therewith.

In block 500, neural network 300 (e.g., implemented by logic device 168) is trained to detect human faces and characteristics affecting human circadian rhythms (e.g., age, gender, and/or other characteristics). For example, as discussed, this may be performed by providing thermal or visible light images of known human faces with known characteristics to neural network 300. Following the training of block 500, neural network will be able to detect human faces and the characteristics in newly received images.

In block 505, logic device 168 obtains one or more circadian rhythm models corresponding to a moving average or median of expected body temperatures and expected body temperature variations associated with persons with particular characteristics (e.g., the circadian rhythm models may be categorized into various sub-classes associated with particular characteristics). For example, persons having an age in a particular range (e.g., 40 to 49 years old) and gender (e.g., female) may exhibit an expected body temperature and an expected variation in the expected body temperature associated with phases of the daily circadian rhythm cycle, while persons having a different age (e.g., 30 to 39 years old) and gender (e.g., male) may exhibit a different expected body temperature and a different expected variation. As a result, a large number of different circadian rhythm models may be provided, each corresponding to expected body temperatures and expected body temperature variations associated with any desired combination of age, gender, and/or other characteristics.

In some embodiments, such circadian rhythm models may be provided to logic device 168. In some embodiments, logic device 168 may adjust, generate, and/or otherwise provide the circadian rhythm models based on processing performed on images. For example, in some embodiments, after a thermal image 400 is processed to determine characteristics of person 192 and one or more temperature values are extracted from the thermal image corresponding to a particular time of day (e.g., a phase of the person's 192 circadian rhythm) in subsequent blocks of FIG. 5, the association between the person's characteristics and temperature values may be used to update circadian rhythm models corresponding to the person's 192 combination of characteristics. For example, such information may be used to adjust the expected body temperatures and expected body temperature variations associated with the relevant circadian rhythm models for the person's 192 combination of characteristics.

In block 510, imager 164 begins capturing thermal images 400 of scene 190 including person 192. For example, thermal image 400 of FIG. 4 represents a thermal image captured in block 510.

In block 515, neural network 300 of logic device 168 processes thermal image 400 to detect face 420 of person 192. For example, in some embodiments, imager 164 may be positioned to capture thermal images of persons 192 passing through scene 190. Thus, in some embodiments, imager 164 may continue to repeatedly capture and process thermal images 400 in blocks 510 and 515 until face 420 of person is detected. In some embodiments, logic device 168 may convert thermal image 400 to a visible light image which is processed to detect face 420.

In block 520, neural network 300 further processes thermal image 400 to detect characteristics of person 192 that may affect their circadian rhythm. As discussed, such characteristics may include age, gender, and/or other characteristics. In some embodiments, logic device 168 may convert thermal image 400 to a visible light image which is processed to detect the characteristics.

In block 525, logic device 168 selects a circadian rhythm model corresponding to the detected characteristics of person 192. In this regard, as discussed in relation to block 505, different circadian rhythm models may be available, each being associated with a particular combination of characteristics. Thus, by selecting the corresponding circadian rhythm model for person 192 in block 525, an expected body temperature and expected variation in such body temperature may be determined for person 192 from the selected circadian rhythm model.

In block 530, logic device 168 detects (e.g., measures) a time and any additional environmental conditions that may affect the body temperature of person 192. For example, logic device 168 may receive the time from clock 179 to identify the phase of the determined circadian rhythm model (e.g., corresponding to the phase within the circadian rhythm model experienced by person 192 at the time thermal image 400 was captured). Additional environmental conditions (e.g., ambient temperature, humidity, and/or other data) that may affect body temperature in some cases may also be determined in block 530, such as data from other sensors 180 and/or other components 182 as appropriate.

In block 535, logic device 168 determines an expected body temperature for person 192 using the selected circadian rhythm model and the time. For example, in some embodiments, the time may be associated with a weight factor applied to an overall expected body temperature provided by the circadian rhythm model that is adjusted by the weight factor to account for phase-based variations to obtain the expected body temperature of person 192 adjusted for variations. In some embodiments, the circadian rhythm model may include a plurality of different expected body temperatures associated with different phases of the circadian rhythm cycle. In such embodiments, the time may be used to select the phase and thus obtain the expected body temperature of person 192.

In block 540, logic device 168 further adjusts the expected body temperature of person 192 to account for the environmental conditions determined in block 530 as appropriate. For example, if environmental conditions exist that may be expected to affect the body temperature of person 192, the expected body temperature may be adjusted accordingly to compensate for the expected effects of such environmental conditions.

In block 545, logic device 168 extracts one or more temperature values from thermal image 400 corresponding to locations on face 420 of person 192. For example, in some embodiments, logic device 168 may extract temperature values from one or more of locations 430, 440, 450, and/or others which correlate to body temperature.

In block 550, logic device 168 compares one or more of the extracted temperature values with the expected body temperature determined in block 535 and/or 540 to determine whether the extracted temperature values are elevated in relation to the expected body temperature. In some embodiments, logic device 168 may compare a single extracted temperature value that correlates to body temperature. In some embodiments, logic device 168 combine multiple temperature values (e.g., by calculating an average or median) to provide a combined extracted temperature value correlating to body temperature for comparison.

In block 555, logic device 168 determines whether the extracted temperature value corresponds to an elevated body temperature value. For example, in some embodiments, logic device 168 may determine whether the extracted temperature value exceeds the expected body temperature value by a sufficient threshold (e.g., greater than 1 degree C., 2 degrees C., or other threshold). If the threshold is exceeded, then logic device 168 determines that person 192 exhibits an elevated body temperature (e.g., person 192 exhibits an elevated body temperature condition) and the process continues to block 560. Otherwise, the process continues to block 565.

In block 560, logic device 168 generates a notification regarding the detected elevated body temperature. For example, in various embodiments, logic device 168 may generate a visible and/or audible notification in the form of text, icons, colors, flashing lights, sounds, alarms, and/or other types notifications to be provided using the various components of imaging system 100 as appropriate.

In block 565, logic device 168 performs a fever analysis by processing a plurality of temperature values previously extracted in block 545. For example, the fever analysis of block 565 can provide a further confirmation (e.g., verification) of the existence or non-existence of an elevated body temperature, regardless of whether an elevated body temperature was previously detected in block 555. As discussed, such an approach can be particularly useful to detect a fever in cases where a person's 192 body temperature falls outside of expected circadian rhythm body temperature variations associated with other persons having the same or similar shared characteristics.

Accordingly, in block 565, logic device 168 may process the extracted temperature values in one or more ways to detect a fever. In one embodiment, a fever may be detected if temperature values extracted from inner canthus locations 430 and 440 exhibit a difference less than a fixed value (e.g., less than 0.2 degrees C.). In another embodiment, a fever may be detected if temperature values extracted from one of inner canthus locations 430 or 440 and oral region 450 exhibit a difference less than another fixed value (e.g., less than 0.5 degrees C.). In another embodiment, a fever may be detected if an absolute value of the average extracted temperature values of both inner canthus regions 430 and 440 and oral region 450 is above a threshold temperature (e.g., 37 degrees C.).

In block 570, logic device 168 determines whether a fever has been detected using the conditions set forth in block 565. If a fever is detected, then the process continues to block 575. Otherwise, the process returns to block 505.

In block 575, logic device 168 generates a notification regarding the detected fever, for example, in a manner as similarly discussed with regard to block 560. Thereafter, the process of FIG. 5 returns to block 505.

Upon returning to block 505 (e.g., from either block 570 or block 575), the process of FIG. 5 repeats blocks 505 to 575 as appropriate to capture and process additional thermal images to detect elevated body temperature and fevers for additional persons 192 in scene 190. Also, as block 505 is repeated, logic device 168 may update one or more of the circadian rhythm models using the detected characteristics, extracted temperatures, elevated body temperature detections, and/or fever detections determined in the previous iteration of any of blocks 520 to 575. For example, appropriate circadian rhythm models may be updated with temperature values extracted from thermal image 400 to improve the accuracy of expected body temperatures and expected body temperature variations used by the circadian rhythm models to improve their accuracy using real world real time measurements provided by thermal image 400.

Other embodiments are also contemplated. Although the process of FIG. 5 has been discussed in relation to thermal images, visible light images may be used for certain processing. For example, in some embodiments, the thermal images may be converted to visible light images (e.g., visible light representations of the thermal images) which are processed for purposes of performing face detection (e.g., block 515) and/or characteristics detection (e.g., block 520). In some embodiments, visible light images concurrently captured with the thermal images (e.g., using an additional visible light imager) may be used for such processing. In yet another embodiment, the features discussed herein may be used to detect the occurrence and stage of a menopause condition that may be present in person 192. For example, menopause occurrence and its associated stage may be one or more characteristics determined in block 520 and associated with one or more sub-classes of circadian rhythm models.

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the spirit of the present disclosure. Where applicable, the various hardware components and/or software components set forth herein can be separated into sub-components comprising software, hardware, or both without departing from the spirit of the present disclosure. In addition, where applicable, it is contemplated that software components can be implemented as hardware components, and vice-versa.

Software in accordance with the present disclosure, such as program code and/or data, can be stored on one or more computer readable mediums. It is also contemplated that software identified herein can be implemented using one or more general purpose or specific purpose computers and/or computer systems, networked and/or otherwise. Where applicable, the ordering of various steps described herein can be changed, combined into composite steps, and/or separated into sub-steps to provide features described herein.

Embodiments described above illustrate but do not limit the invention. It should also be understood that numerous modifications and variations are possible in accordance with the principles of the present invention. Accordingly, the scope of the invention is defined only by the following claims.

What is claimed is:

1. A method comprising:
receiving a thermal image;
processing the thermal image to detect a person's face and a characteristic associated with the person;
selecting a circadian rhythm model associated with the detected characteristic;
determining an expected body temperature using the circadian rhythm model;
extracting a temperature associated with the person's face from the thermal image; and
comparing the extracted temperature with the expected body temperature to detect an elevated body temperature condition.

2. The method of claim 1, further comprising receiving a time associated with the thermal image, wherein the determining comprises using the time to identify a phase of the circadian rhythm model.

3. The method of claim 2, wherein the determining comprises selecting the expected body temperature corresponding to the phase from a plurality of expected body temperatures corresponding to a plurality of phases of the circadian rhythm model.

4. The method of claim 2, wherein the determining comprises adjusting the expected body temperature in response to the identified phase.

5. The method of claim 1, wherein the circadian rhythm model corresponds to a sub-class associated with the characteristic, the method further comprising updating the circadian rhythm model corresponding to the sub-class using the extracted temperature to improve accuracy of the circadian rhythm model.

6. The method of claim 1, further comprising determining a difference between extracted temperatures from the thermal image to detect a fever condition associated with the person, wherein the extracted temperatures comprise a temperature of a left inner canthus of the person, a temperature of a right inner canthus of the person, and/or a temperature of an oral region of the person.

7. The method of claim 6, further comprising generating a notification of the elevated body temperature condition and/or the fever condition.

8. The method of claim 1, wherein the processing is performed using the thermal image and a visible light image to detect the characteristic, wherein the characteristic is a first characteristic comprising an age associated with the person, wherein the method further comprises processing the thermal image and the visible light image to detect a second characteristic comprising a gender associated with the person.

9. The method of claim 1, wherein the processing is performed using the thermal image and a visible light image to detect the characteristic, wherein the characteristic is an age and/or a gender of the person.

10. The method of claim 1, wherein:
the method is performed by a portable thermal camera;
the processing is performed by a neural network using the thermal image and a visible light image; and
the method further comprises:
training the neural network to detect the face and the characteristic,
stabilizing the thermal image, and
averaging spatial and temporal pixel values of a plurality of thermal images to improve accuracy of the extracted temperature, wherein the extracted temperature is associated with an inner canthus of the person's face.

11. A system comprising:
a thermal imager; and
a logic device configured to:
operate the thermal imager to capture a thermal image,
process the thermal image to detect a person's face and a characteristic associated with the person,
select a circadian rhythm model associated with the detected characteristic,
determine an expected body temperature using the circadian rhythm model,
extract a temperature associated with the person's face from the thermal image, and
compare the extracted temperature with the expected body temperature to detect an elevated body temperature condition.

12. The system of claim 11, wherein the logic device is configured to receive a time associated with the thermal image and use the time to identify a phase of the circadian rhythm model.

13. The system of claim 12, wherein the logic device is configured to select the expected body temperature corresponding to the phase from a plurality of expected body temperatures corresponding to a plurality of phases of the circadian rhythm model.

14. The system of claim 12, wherein the logic device is configured to adjust the expected body temperature in response to the identified phase.

15. The system of claim 11, wherein the circadian rhythm model corresponds to a sub-class associated with the characteristic, wherein the logic device is configured to update the circadian rhythm model corresponding to the sub-class using the extracted temperature to improve accuracy of the circadian rhythm model.

16. The system of claim 11, wherein the logic device is configured to determine a difference between extracted temperatures from the thermal image to detect a fever condition associated with the person, wherein the extracted temperatures comprise a temperature of a left inner canthus of the person, a temperature of a right inner canthus of the person, and/or a temperature of an oral region of the person.

17. The system of claim 16, wherein the logic device is configured to generate a notification of the elevated body temperature condition and/or the fever condition.

18. The system of claim 11, wherein the logic device is configured to process the thermal image and a visible light image to detect the characteristic, wherein the characteristic is a first characteristic comprising an age associated with the person, wherein the logic device is configured to process the thermal image and the visible light image to detect a second characteristic comprising a gender associated with the person.

19. The system of claim 11, wherein the logic device is configured to process the thermal image and a visible light image to detect the characteristic, wherein the characteristic is an age and/or a gender of the person.

20. The system of claim 11, wherein:
the system is a portable thermal camera;
the logic device comprises a neural network configured to process the thermal image and a visible light image to detect the person's face and the characteristic;
the neural network is configured to be trained to detect the face and the characteristic; and
the system is configured to:
stabilize the thermal image, and
average spatial and temporal pixel values of a plurality of thermal images to improve accuracy of the extracted temperature, wherein the extracted temperature is associated with an inner canthus of the person's face.
